(19) United States
(12) Patent Application Publication
(10) Pub. No.: US 2025/0352604 A1
(43) Pub. Date: Nov. 20, 2025

(54) COMPOSITIONS AND METHODS FOR IMPROVING SLEEP AND MANAGING STRESS

(71) Applicant: KARALLIEF INC., Boston, MA (US)

(72) Inventor: Krishna Rajendran, Boston, MA (US)

(21) Appl. No.: 18/667,671

(22) Filed: May 17, 2024

(51) Int. Cl. A61K 36/84; A61K 31/192; A61K 31/216; A61K 31/7048; A61K 36/53; A61K 36/71; A61K 36/725; A61K 36/86; A61P 25/00

(52) U.S. Cl. CPC A61K 36/84; A61K 31/192; A61K 31/216; A61K 31/7048; A61K 36/53; A61K 36/71; A61K 36/725; A61K 36/86; A61P 25/00

(57) ABSTRACT

The present invention is directed to a composition including the following combination of ingredients: Valeriana officinalis extract, Passiflora incarnata extract, Ocimum sanctum extract, Ziziphus jujuba extract, Rosmarinus officinalis extract, and Nigella sativa extract. The compositions can be used in methods of improving or promoting sleep and managing and reducing stress.

### BACKGROUND

### Technical Field of the Invention

The technical field of this invention relates to natural product compositions and formulations for improving sleep and managing stress.

### SUMMARY DISCLOSURE OF THE INVENTION

The present invention is directed to a composition including the following combination of ingredients: Valeriana officinalis extract, Passiflora incarnata extract, Ocimum sanctum extract, Ziziphus jujuba extract, Rosmarinus officinalis extract, and Nigella sativa extract. The compositions can be used in methods of improving sleep and managing stress in a human.

Other features and aspects will be apparent from the following detailed description, the drawings, and the claims.

### BRIEF DESCRIPTION OF THE DRAWINGS

FIG. 1 shows a design outline of the trial.

FIG. 2 shows a Disposition of Subjects.

FIG. 3A shows an active compound of the disclosure.

FIG. 3B shows another active compound of the disclosure.

FIG. 3C shows another active compound of the disclosure.

FIG. 4 shows another active compound of the disclosure.

FIG. 5 shows another active compound of the disclosure.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DISCLOSURE OF THE INVENTION

The following detailed description is provided to assist the reader in gaining a comprehensive understanding of the methods, products, and/or systems, described herein. However, various changes, modifications, and equivalents of the methods, products, and/or systems described herein will be apparent to an ordinary skilled artisan.

The compositions of the present invention are directed to a product containing the following combination of ingredients: Valeriana officinalis extract, Passiflora incarnata extract, Ocimum sanctum extract, Ziziphus jujuba extract, Rosmarinus officinalis extract, and Nigella sativa extract.

### MODES FOR CARRYING OUT THE INVENTION

In one embodiment, the compositions of the present invention are directed to a product containing about 16% to about 24% by weight of Valeriana officinalis extract, about 12% to about 18% by weight of Passiflora incarnata extract, about 12% to about 18% by weight of Ocimum sanctum extract, about 12% to about 18% by weight of Ziziphus jujuba extract, about 12% to about 18% by weight of Rosmarinus officinalis extract, and about 16% to about 24% by weight of Nigella sativa extract.

In a preferred embodiment, the product may contain about 20% by weight of Valeriana officinalis extract, about 15% by weight of Passiflora incarnata extract, about 15% by weight of Ocimum sanctum extract, about 15% by weight of Ziziphus jujuba extract, about 15% by weight of Rosmarinus officinalis extract, and about 20% by weight of Nigella sativa extract.

In an embodiment, the compositions can consist essentially of Valeriana officinalis extract, Passiflora incarnata extract, Ocimum sanctum extract, Ziziphus jujuba extract, Rosmarinus officinalis extract, and Nigella sativa extract. In this case, it is understood that a composition consisting essentially of the above extracts can include further components that are commonly included in pharmaceutical or dietary supplement compositions such as carriers, diluents, adjuvants, solubilizing agents, suspending agents, fillers, surfactants, an antimicrobial agent, a preservative, a viscosity modifier, a thixotropy modifier, a wetting agent, an emulsifier, etc.

In another embodiment, the compositions consist of Valeriana officinalis extract, Passiflora incarnata extract, Ocimum sanctum extract, Ziziphus jujuba extract, Rosmarinus officinalis extract, and Nigella sativa extract. In this case, each extract is included at a purity level that are commercially available. The consisting of transitional phrase would not exclude common impurities, side products, and the like, that could be present due to the extraction or manufacturing process.

The compositions can be in the form of a liquid, gel, suspension, dispersion, solid, emulsion, aerosol, powders, tablets, capsules, pills, liquids, suspensions, dispersions or emulsions. Also, the compositions disclosed herein can be in a form suitable for dilutions. Similarly, the compositions can be in the form of a powder, cream, paste, gel or solid. In one preferred embodiment, the compositions are included in powder form in a capsule. The compositions can include carriers, diluents, adjuvants, solubilizing agents, suspending agents, fillers, surfactants, an antimicrobial agent, a preservative, a viscosity modifier, a thixotropy modifier, a wetting agent, an emulsifier, etc. These terms are used consistent with current usage in the pharmaceutical arts as evidenced, for example, by Remington's Pharmaceutical Sciences, Sixteenth Edition, E. W. Martin (Mack Publishing Co., Easton, Pa., 1980); and Remington: Essentials of Pharmaceutics, Pharmaceutical Press; 1st edition (Apr. 1, 2013).

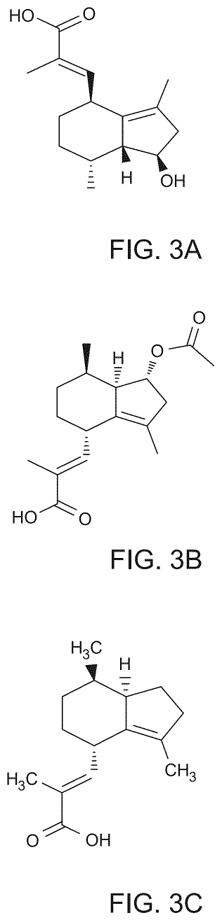

An active compound of the composition can include the active compounds: (E)-3-[(1R,4S,7R,7aR)-1-hydroxy-3,7-dimethyl-2,4,5,6,7,7a-hexahydro-1H-inden-4-yl]-2-methylprop-2-enoic acid shown in FIG. 3A, (E)-3-[(1R,4S,7R,7aR)-1-acetyloxy-3,7-dimethyl-2,4,5,6,7,7a-hexahydro-1H-inden-4-yl]-2-methylprop-2-enoic acid shown in FIG. 3B, and (E)-3-[(4S,7R,7aR)-3,7-dimethyl-2,4,5,6,7,7a-hexahydro-1H-inden-4-yl]-2-methylprop-2-enoic acid shown in FIG. 3C.

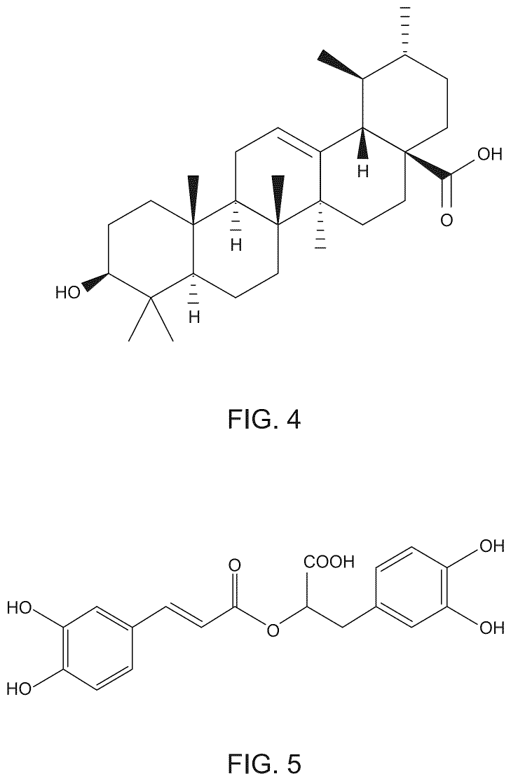

An active compound of the composition can include (1S,2R,4aS,6aR,6aS,6bR,8aR,10S,12aR,14bS)-10-hydroxy-1,2,6a,6b,9,9,12a-heptamethyl-2,3,4,5,6,6a,7,8,8a,10,11,12,13,14b-tetradecahydro-1H-picene-4a-carboxylic acid shown in FIG. 4.

An active compound of the composition can include Vitexin (C₂₁H₂₀O₁₀, molecular weight=432.4).

An active compound of the composition can include (2R)-3-(3,4-dihydroxyphenyl)-2-[(E)-3-(3,4-dihydroxyphenyl) prop-2-enoyl]oxypropanoic acid shown in FIG. 5.

The subject compositions of this invention can be used in methods of improving sleep and managing stress. Methods of use generally include the steps of administering a composition of the present invention to a patient in need thereof to manage stress and/or improve sleep. The methods may increase total sleep time and sleep efficiency and provide a decrease in sleep onset latency, WASO minutes, i.e., Wake After Sleep Onset minutes i.e. how long the subject is awake (as measured by the Actiwatch) after the subject has first fallen asleep, and frequency of awakenings. The methods can also provide a decrease in PSS scores thus providing effective reduction in stress. The methods can provide a reduction in Serum Cortisol and Hs CRP levels.

Dosage be from 100 mg to 1000 mg per day. In a preferred embodiment, the dosage can be 500 mg per day. For example, the administration can include providing a pill, capsule, or tablet, once per day orally at a prescribed dosage.

### Glossary

KaraCalm™ is a product containing the combination of ingredients: Valeriana officinalis extract, Passiflora incarnata extract, Ocimum sanctum extract, Ziziphus jujuba extract, Rosmarinus officinalis extract, and Nigella sativa extract.

Valeriana officinalis extract is an extract from a perennial flowering plant native to Europe and Asia. See e.g., Valerian (herb), Wikipedia, the free encyclopedia, of record.

Passiflora incarnata extract, commonly known as maypop, purple passionflower, true passionflower, wild apricot, and wild passion vine, is an extract from a Passiflora incarnata perennial with climbing or trailing stems. See, e.g., Passiflora incarnata, Wikipedia, the free encyclopedia, of record.

Ocimum sanctum extract, aka Ocimum tenuiflorum, aka holy basil or tulsi, is an extract from an aromatic perennial plant in the family Lamiaceae. See e.g., Ocimum tenuiflorum, Wikipedia, the free encyclopedia, of record.

Ziziphus jujuba extract, aka jujuba, also called red date, Chinese date, and Chinese jujube, is a species in the genus Ziziphus in the buckthorn family Rhamnaceae. See e.g., Jujube, Wikipedia, the free encyclopedia, of record.

Rosmarinus officinalis, aka Salvia Rosmarinus, and rosemary, is an extract from a shrub with fragrant, evergreen, needle-like leaves and white, pink, purple, or blue flowers, native to the Mediterranean region. See e.g., Rosemary, Wikipedia, the free encyclopedia, of record.

Nigella sativa extract, aka, black caraway, black cumin, nigella, kalonji, and charnushka is an extract from an annual flowering plant in the family Ranunculaceae. See e.g., Nigella sativa, Wikipedia, the free encyclopedia, of record.

The term Extracts as used herein, is used consistently with the term as known in the natural products arts and are obtained from natural products as described, for example, Techniques for Extraction and Isolation of Natural Products: A Comprehensive Review, Zhang et al., Chin Med (2018) 13:20, https://doi.org/10.1186/s13020-018-0177-x.

Vitexin is an apigenin flavone glucoside. See Vitexin, Wikipedia, the free encyclopedia, of record.

A sleep disorder or somnipathy is a medical disorder of an individual's sleep patterns. See e.g., Sleep Disorder, Wikipedia, the free encyclopedia, of record. Sleep disorders can be classified into dyssomnias, parasomnias, circadian rhythm sleep disorders involving the timing of sleep, and other disorders such as those caused by medical or psychological conditions. When a person struggles to fall asleep or stay asleep with no obvious cause, it is referred to as insomnia. Others sleep disorders include sleep apnea, narcolepsy and hypersomnia, sleeping sickness, sleepwalking, and night terrors.

A Stress-related disorder is a category of mental disorder. See Stress-Related Disorders, Wikipedia, the free encyclopedia, of record. Stress-related disorders include Obsessive-Compulsive Disorder (OCD), Post-Traumatic Stress Disorder (PTSD) PTSD, Complex Post-Traumatic Stress Disorder (CPTSD), and adjustment disorder.

Administration of the inventive compounds as described herein include oral administration, injection routes, e.g., subcutaneous, intramuscular, intravenous, intrathecal, sublingual, and buccal, rectal, vaginal, ocular, otic, nasal, inhalation, nebulization, cutaneous, transdermal, and using tablets, capsules, or enteric coatings. See e.g., Drug Administration, the Merck manual (consumer version), reviewed/revised June 2022/modified September 2022.

A tablet or pill is a pharmaceutical/dietary supplement oral dosage form (oral solid dosage, or OSD) or solid unit dosage form. See e.g., tablet (pharmacy), Wikipedia, the free encyclopedia, of record.

A capsule is a pharmaceutical/dietary supplement dosage form used to enclose a medicine or composition in a relatively stable shell allowing the composition to be taken orally or as a suppository. A capsule is generally a small container with medicine/dietary supplement inside that can be swallowed or a gelatin shell enclosing medicine/dietary supplement. Capsules as used herein include hardshell capsules and softshell capsules as known in the art. Hardshell capsules usually contain dry, powdered ingredients. Softshell Capsules usually use a gelatin shell surrounding the composition. Softshell capsules can be, for example, used to hold liquid compositions. See e.g. capsule (pharmacy), Wikipedia, the free encyclopedia, of record.

### Example

A randomized, double-blind, placebo-controlled clinical study was done to assess the efficacy and safety of the present invention to manage stress and improve sleep in adults. The primary endpoint of this study was to assess the change in Sleep analysis by using ActiWatch and Perceived Stress Scale (PSS) scores from baseline to the end of the study period. The sample size was 60 subjects who were distributed equally into two study arms of the present compositions or placebo. The total duration of the study was 56 days. Each subject needed to report nine times at the clinical center including screening and randomization day.

This study was designed to compare efficacy of the present novel polyherbal formula versus a placebo. The present compositions serve as a positive control and placebo group serves as negative control. Comparison with placebo group provided clear information on efficacy of the present invention without the placebo effect.

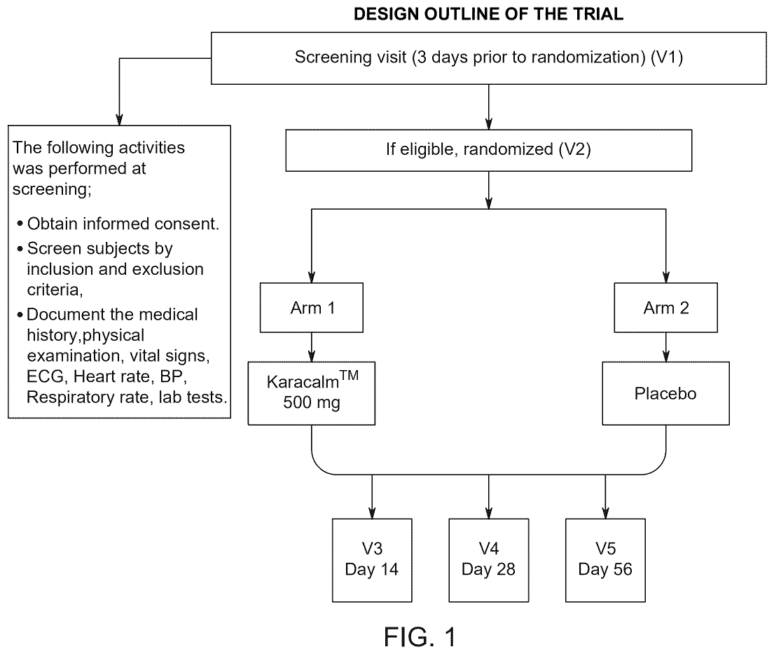

FIG. 1 shows the design outline of the trial.

TABLE 3
EVENTS SCHEDULE CHART

| | | Visit 2 | Visit 3 | Visit 4 | Visit 5 | Visit 6 | | Visit 8 | Visit 9 |
|---|---|---|---|---|---|---|---|---|---|
| | Visit 1 | Day −1/ | Day 0/ | Day 3/ | Day 9/ | Day 13/ | Visit 7 | Day 51/ | Day 55/ |
| Visits* | Day −3 | Day 0 | Day 1 | Day 4 | Day 10 | Day 14 | Day 28 | Day 52 | Day 56 |
| Informed consent | X | — | — | — | — | — | — | — | — |
| Eligibility criteria | X | — | — | — | — | — | — | — | — |
| Demographic data | X | — | — | — | — | — | — | — | — |
| Medical history | X | — | — | — | — | — | — | — | — |
| Physical | X | — | X | — | — | X | X | — | X |
| examination | | | | | | | | | |
| Vital signs | X | — | X | — | — | X | X | — | X |
| Concomitant | | — | X | — | — | X | X | — | X |
| medications | | | | | | | | | |
| Safety-Laboratory | X | — | — | — | — | — | — | — | X |
| investigations-CBC, | | | | | | | | | |
| LFT, RFT, ECG, | | | | | | | | | |
| Lipid profile | | | | | | | | | |
| HIV, RBS and UPT | X | — | | — | — | — | — | — | — |
| Bio markers | — | — | X | — | — | X | — | — | X |
| Cortisol, hsCRP 3 | | | | | | | | | |
| times | | | | | | | | | |
| Anti-stress and | X | — | X | — | — | X | X | — | X |
| Sleep scales # | | | | | | | | | |
| Randomization | — | — | X | — | — | — | — | — | — |
| *IP reconciliation/ | — | — | X | — | — | X | X | — | X |
| dispensing | | | | | | | | | |
| Adverse Events | — | — | — | — | — | X | X | — | X |
| End of treatment | — | — | — | — | — | | | — | X |
| End of study | — | — | — | — | — | — | — | — | X |
| Actigraphy in 60 | X | X | X | X | X | X | — | X | X |
| subjects each - | | | | | | | | | |
| 3 times | | | | | | | | | |

Each visit had Window Period: +/−3 Days

### Selection of Participants

Participants must have met all the inclusion criteria to participate in this study:

Male and Female healthy adult subjects ranging in age from 18 to 54 years; Adults Subjects willing to provide a written Informed Consent; Free of psychiatric conditions; Could read and write English.

Patients who met any one of the following criteria at baseline were not considered for the study:

Subjects with insomnia, sleep disorders, or chronic stress; Chronic alcoholics (more than 2 standard pegs/day); Subjects with known Hypertension and other diseases of the cardiovascular system; Subjects with known Liver diseases, Kidney diseases, Psychiatric diseases, Epilepsy and/or with any other relevant diseases; Subjects with the intention of non-compliance to the study-protocol; Subject participating in another clinical trial or has received any IP within 90 days prior to Visit 1 (Screening); Retraction of the written informed consent; Subjects currently taking medications other than oral contraceptive pill; Participants on hepatotoxic medications like antitubercular medication, antiviral medication, paracetamol etc.; Pregnant, attempting to conceive, or lactating women; Individuals with acute narrow-angle glaucoma, prostate hypertrophy, cardiovascular, endocrine or renal disease, or another chronic disease that could affect stress/anxiety or restrict normal, daily function were also ineligible to participate in the study; Individuals who currently, or in the past 6 months, suffered from any diagnosable mental-health disorder (as assessed by the Mini International Neuropsychiatric Interview 6.0) or were taking a psychotropic medication or other herbal preparation were also excluded from participating in the study.

Subjects who did not meet inclusion/exclusion criteria were considered as screen failure. Participating subjects were allowed to withdraw from the study at any time without the need to justify his/her decision even after undergoing the consenting process (consent withdrawal). No subjects were discontinued from the study due to non-compliance with medication, protocol violation, worsening of disease or tolerability, Adverse Events (AE) or Serious Adverse Events (SAE).

### Study Interventions

Eligible subjects were randomly allocated to either of the study arms in accordance with the Randomization code mentioned in the study product containers. The same had been documented into the Randomization record. The study product contains one of the following regimens packed in strips with secondary packing: Group A (n=Recruiting 30 subjects): IP KaraCalm™; Group B (n=Recruiting 30 subjects) placebo. The Pharmacist prepared labels for IP dispensing containers. Subjects were advised to take one capsule once a day orally, after dinner for 56 days.

KaraCalm™ 500 mg capsules were administered orally once daily. The KaraCalm™ product contained about 20% by weight of Valeriana officinalis extract, about 15% by weight of Passiflora incarnata extract, about 15% by weight of Ocimum sanctum extract, about 15% by weight of Ziziphus jujuba extract, about 15% by weight of Rosmarinus officinalis extract, and about 20% by weight of Nigella sativa extract.

Study medication was stored in the site in a secure place at room temperature. Product accountability was maintained. Unused medication if any, was returned to the sponsor at the end of the study. Daily temperature recording was entered in a temperature log which was provided to every investigator.

The sponsor was responsible for the supply of all the Investigational Products (IPs) in properly labelled packs and proper storage conditions. All the IP were packed in white HDPE bottles. Three bottles were packed with 62 capsules for visit 3 (16 Capsules) and visit 6 (16 Capsules) and Visit 7 (30 Capsules). The study medications were packed according to the assigned randomization number.

Upon receipt of the investigational product supplies, an inventory was maintained, and investigational product receipt log filled out and signed by the person accepting the shipment. The shipment was verified with respect to counts and status and all the items were noted in the shipment inventory. The investigator was requested to notify study sponsor of any damaged or unusable study supplements (if any) that were supplied to the investigator's site. No damaged or unusable investigational product in each shipment (KaraCalm™ and placebo) was received by the investigator.

All the study medications were stored at room temperature in a cool and dry place. Each subject was provided one bottle of Investigational Product (IP) during visits V3, V6 and V7. Subjects were asked to take one capsule a day after dinner. Any unused IP was returned to the investigator or designated staff during the visits. This reconciliation was logged on the investigational product reconciliation form and signed and dated by the study team.

The investigator maintained 100% accountability for all study medication received and dispensed during his or her entire participation in the study. Proper investigational product accountability includes but is not limited to: Frequently verifying that actual inventory matches documented inventory; Verifying that the log is completed for the investigational product lot used to prepare each dose; Verifying that all containers used are documented accurately on the log; Verifying that required fields are completed accurately and legibly.

The investigator maintained a current inventory (Investigational Product Accountability Log) of all IP delivered to the site, inventory at the site, and subjects' use records, which accurately always reflected the investigational product accountability of the IP. The accountability log contained the following information-protocol number, name of investigator, site identifier and number, date and amount dispensed, and the date and amount returned to the site by the subject, including the initials of the person dispensing and receiving the IP. There was a separate entry for each subject to whom the IP was dispensed.

Prior to site closure or at appropriate intervals, a representative from the sponsor or its designee performed clinical study material accountability and reconciliation before clinical study materials are returned to the sponsor or its designee for destruction. The study materials were returned to the sponsor for destruction. The investigator retained the original documentation regarding clinical study material accountability and return.

The sealed packs of KaraCalm™ (containers) were provided by the sponsor to the clinical site. Investigator or the designated staff, who received the IP maintained inventory logs and reconciliation logs for individual supplies. Investigator prescribed KaraCalm™ as per the protocol-based randomization to the subjects. The randomization codes were generated by computer using permuted block design and the block size selected was known only to the statistician until the statistical analysis was completed.

The randomization code list was maintained by Sponsor's designee. It was provided to the CRO and study sites in a concealed envelope. The site coordinator intimated the sponsor's designee after allotting the randomization code to the eligible subjects. At no point in the study, the code was broken, or un-blinded study product was administered to any subject.

All study subjects, investigators and sponsor's personnel remained blinded to study medication assignment. The Investigator was given right to break the blind in special situations such as: 1. Treatment emergent SAE's; 2. To protect the safety of the patient.

It was made clear that the investigator should try to contact Green Chem, Bangalore designee, in such scenario, before breaking the code. However, it was communicated that in the event of un-blinding, as result of a serious adverse event, the sponsor designee and the IRB/IEC must be notified immediately by the investigator.

All study subjects, investigators and Green Chem personnel remain blinded to investigational product assignment but restricted access to the codes in the event of an emergency are available through the sponsor designated person implementing randomization/blinding.

Each subject was administered with KaraCalm™ as per the randomization. Subjects were prescribed to consume as 500 mg capsules once daily orally for 56 days. The consumption of the IP was documented by the subjects into a daily diary and compliance card provided to them.

KaraCalm™ was presented in 500 mg capsules and was taken by subjects once daily orally for 56 days through oral supplementation only. The medications were dispensed during visit 3, visit 6 and visit 7 accordingly. The study medications had been packed according to the assigned randomization number. Subjects entered the consumption of study supplement in their diary cards provided to them. Any unused study supplement was returned to the investigator or designated staff during the visits. Either the investigator or the designated staff verified the Subject daily diary and compliance card and reconciled the provided study medication to the subjects. This reconciliation was logged onto the investigational product reconciliation form and signed and dated by the study team.

This was a randomized, double-blind, placebo-controlled study. The investigator, subjects and the biostatistician were blinded for the treatment provided to each participant.

A complete medical history was taken during the screening period and throughout the study period on all visits. The medical history was recorded on the appropriate section of the Case Report Forms (CRF) which included all significant past medical or surgical procedures (if any) as well as previous and current co-existent diseases (if any) for individual subjects. Other nutritional supplements for sleep and stress were avoided during the participation in the study. Subjects once enrolled did not take any concomitant medications. Medications that did not affect the clinical effectiveness of KaraCalm™ may be permitted as per PI discretion. Based on investigators advice, use of rescue medication (if any) was recorded in the source document and case report form. Subjects who require rescue medication were considered a treatment failure.

The investigator maintained 100% accountability for all study medication received and dispensed throughout the study period. The investigator maintained an up-to-date inventory (Investigational Product Accountability Log) of all IP delivered to the site, inventory at the site, and subjects' use records. A separate entry was made into the log for the IP that was dispensed.

Six subjects from each of the two groups dropped out due to Lost to Follow up.

A complete medical history was recorded during the screening period and review of concomitant medication was done throughout the study period on all visits. The medical history was documented on the appropriate section of the CRF. Significant past medical or surgical procedures as well as previous and current co-existent diseases (if any) were included. It also included relevant medical history for the following body systems: head, neck, eyes, ENT, respiratory, pulmonary, cardiovascular, gastrointestinal, endocrine, hematological, dermatological, renal, neurological, musculoskeletal, psychological/psychiatric etc. Past and present medical history was noted along with the past and present medications (if any). These were recorded in the appropriate section of the CRF.

A complete physical examination of each subject was conducted at all visits. Vital signs (heart rate, blood pressure, temperature, respiratory rate and pulse rate) and weight were recorded at all visits.

Each subject underwent the clinical laboratory tests, performed at screening and at selected follow up visits. Urine for urinalysis and blood for haematology, biochemistry was collected on screening and end of the study visit.

As the study medication was derived from well-known and widely consumed herbal sources, there was no Adverse Event expected upon their consumption. As these medications comprised of ingredients known for human consumption, specific attention was given to subjects who might not be aware of their specific allergy to these constituents or herbs in general and might develop an allergy while in trial. Those who might be having a known allergy to KaraCalm™ products were excluded from the trial.

There was no severe adverse event reported in this study.

There was no severe adverse event reported in this study that led the investigator/subject to permanently withdraw/discontinue the subject.

The investigator verified and assessed all the reports for the possibility of any abnormal or clinically significant laboratory values. No abnormal or clinically significant laboratory values were found to be possibly related to the study product.

For the purposes of this study, a dosing error was defined as a dose exceeding or less than the number of capsules of study product specified for each dose, or the administration of study product from a bottle set assigned to a different subject. Such occurrences were required to be reported and recorded in the dosing page of the CRF as follows:

Use of study product doses in excess of that specified in the protocol or use of the wrong medication bottle or error in timing or dose should not be recorded as an AE unless there are any associated signs or symptoms.

A dosing error with associated non-serious AEs should be recorded as an AE of the relevant AE forms in the CRF with a notation in the comment field explaining the magnitude of the dosing error. A dosing error with associated SAEs should be recorded in the relevant SAE form.

There was no dosing error reported or recorded in this study.

It was the responsibility of the principal investigator to oversee the safety of the study at his/her site. This safety monitoring included careful assessment and appropriate reporting of AEs as noted above, as well as the construction and implementation of a site data and safety-monitoring plan. Study monitoring included a regular assessment of the number and type of SAEs.

These measurements were widely used for assessment of sleep and stress parameters and are recommended for clinical trials and therapeutic purposes.

The investigator permitted trial-related monitoring, audits, IEC review and regulatory inspection(s), providing direct access to source data and documents. The investigator obtained the prior written consent of the patient or the patient's legally acceptable representative for such direct access.

Study monitors from Contract Research Organization (CRO) conducted site visits on different intervals as defined in the site monitoring visits SOPs to assess the site facilities. The sponsor's designee and CRO informed the investigator and all trial staff of their responsibilities and the procedures for ensuring adequate and correct documentation.

The sponsor's monitor and independent QA auditor conducted periodic data reviews and verification, against the source data, to ensure the quality of the data to be maintained. The investigator was responsible for ensuring the accuracy and credibility of the data by ensuring the process was in place for the review of completed CRFs prior to any inspection by the monitor.

Subject names and addresses were kept confidential and were not included in the database supplied to the sponsor or its designee. Only enrolment number, subject initials, and birth date were recorded on the CRF. If the subject's name appears on any other document collected (e.g., hospital discharge summary), the name was obliterated before the document is transmitted to the sponsor or its designee. The subjects provided their explicit permission for representatives of the sponsor, regulatory authorities, and the IRB/IEC to inspect their medical records to verify the information collected. Subjects were informed that all personal information made available for inspection were handled in the strictest confidence and in accordance with all state, local, and federal data protection/privacy laws. The investigator maintained a subject identification log (enrolment numbers and corresponding subject names) to enable records to be identified.

All information, original records of clinical findings, observations, or other activities e.g., hospital records, clinical and office charts, laboratory notes, memoranda, subjects' diaries or evaluation checklists, Pharmacy dispensing records, recorded data from automated instruments, copies or transcriptions certified after verification are being accurate and complete, microfiches, photographic negatives, microfilm or magnetic media, x-rays, subject files and records were kept at the pharmacy, at the laboratories, and at medico-technical departments involved in the clinical trial pertaining to the subjects in the clinical trial necessary for the reconstruction and evaluation of the trial were considered as source data and were available in source documents.

The paper-based case report form (CRF) was the primary data collection instrument for the study. All the data requested as per the protocol were recorded on to the CRF. All missing data must be explained. If a space on the CRF was left blank because the procedure was not done, or the question was not asked, “N/D” had been marked. If the item was not applicable to the individual case, “N/A” was marked. All entries were printed legibly in black ink. If any entry error has been made, to correct such an error, a single straight line through the incorrect entry was drawn and the correct data above it was entered. All such changes were initialed and dated. No ERASER or WHITE OUTS were used for any ERRORS. For clarification of illegible or uncertain entries, clarification was noted above the item, initialled, and dated.

A copy of all records of the study would be maintained by the investigator for at least the shorter of the following two-time periods:

Two years after approval of the investigational product for the indication for which it was investigated; In situations where no further application is to be filed or an application is not approved for such indication.

The investigator is maintaining a copy of all data collected for each subject treated (including CRFs and source data). In order to assure the accuracy of data collected in the CRF, it was made mandatory that representatives of the sponsor, or designee, as well as representatives of the regulatory authorities, should have direct access to the original source documents (e.g., subject records, subject charts, and laboratory reports).

Investigator maintained a copy of all data collected for each subject treated (including CRFs and source data). In order to assure the accuracy of data collected in the CRF, it was made mandatory that representatives of the sponsor, or designee, as well as representatives of the regulatory authorities should have direct access to original source documents (e.g., subject records, subject charts and laboratory reports). During the review of these documents, the anonymity of the subjects was respected with strict adherence to professional standards of confidentiality. The sponsor reserved the right to terminate the study for refusal of the investigator to supply source documentation of work performed in this clinical study.

In accordance with applicable regulations, Good Clinical Practices (GCP) and the procedures of the sponsor or its designees, the study monitor periodically contacted the site and conducted on-site visits. The extent, nature and frequency of on-site visits were based on enrolment rate and data quality at the site. Through frequent communications (e.g., letter, and telephone), the study monitor ensured that the investigation was conducted according to the protocol and regulatory requirements.

### Analysis and Outcomes

The final statistical analysis and results were depicted for 48 participants at the end of the study. Data was analyzed using IBM SPSS 21. For continuous variables, mean and SD were obtained, and for categorical variables, frequency and percentage. ANOVA with Post Hoc Tukey's for used for intragroup comparison. The test compared Days 2-14 and 56. The independent T test was used to compare Group A (Karacalm™) with Group B (Placebo) at all time intervals (Day 2, Day 14, and Day 56) with a 95% confidence interval.

No protocol deviations were reported during the entire study.

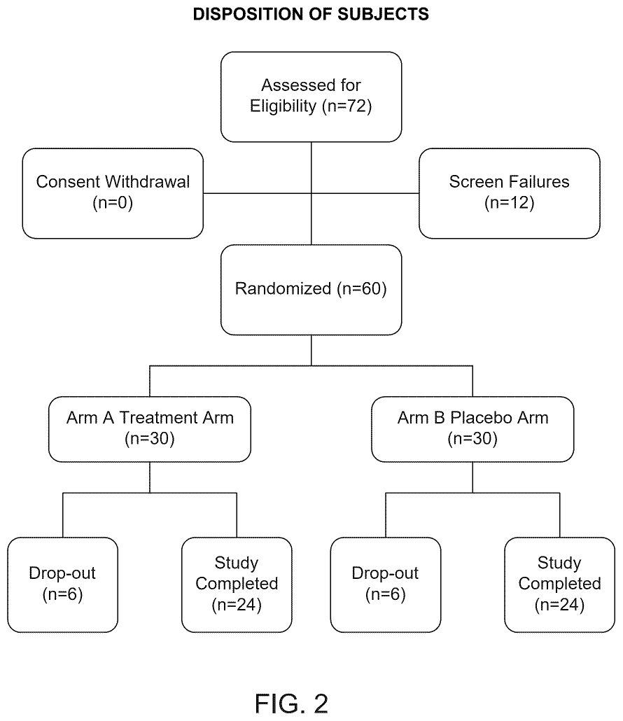

FIG. 2 shows Disposition of Subjects.

TABLE 5
STUDY POPULATION

| Study Population | n |
|---|---|
| Total number of subjects screened | 64 |
| Total number of subjects enrolled & randomized | 60 |
| Intent-to-treat Population | 60 |
| Safety Population | 48 |
| Per- Protocol Population | 48 |

Six subjects from Group A and six from the Group B withdrew due to lost to follow up. The final statistical analysis and results were depicted for 48 participants at the end of the study.

The following demographic and baseline characteristics was summarized by study arm as follows: age, height, weight, medical and medication histories, general physical examination, and serum chemistry. Ordinal and continuous data is presented in the form of descriptive statistics, as number of patients, median, mean, standard deviation, standard error, min-max, 95% confidence interval (95% CI). Categorical data was presented using relative frequencies and their percentage. A summary of baseline demographic data of the subjects is summarized in Table 6, for all the analysis sets.

TABLE 6
SUMMARY OF SUBJECT'S DEMOGRAPHY

| | | Group A | Group B |
|---|---|---|---|
| Variable | Statistics | (n = 24) | (n = 24) |
| Gender | | | |
| Male | N(%) | 11(45.8%) | 11(45.8%) |
| Female | N(%) | 13(54.2%) | 13(54.2%) |
| Age at baseline | N | 24 | 24 |
| | Mean (SD) | 31.3750(9.59081) | 32.2500(8.37828) |
| | (Min, Max) | (21, 53) | (20, 53) |

The mean age in Group A was 31.3750 ± 9.59081 years and in Group B was 32.2500 ± 8.37828 years.

The primary efficacy parameter was to assess changes in baseline to the end of the study period. The main analysis for hypothesis testing conducted by the statistician consisted of ANOVA at baseline (V2), 14 days (V3), 28 days (V4) and 56 days (V5). The results of the ANOVA indicated significant improvements from baseline to each subsequent visit in Group A when compared to Group B.

The results of the study are depicted in the Tables and Figures.

Get up time: Subjects in the KaraCalm™ group experienced a statistically significant increase in their get up time compared to the Placebo group at Day 14 and Day 56, indicating that the KaraCalm™ subjects had improved sleep and were not waking up earlier.

Total Sleep Time: This analysis showed that the participants who were administered Karacalm™ saw a statistically significant increase in their total sleep time (i.e they slept for more time soundly with lesser time awake in bed) at Day 14 and 56, compared to the Placebo group participants (in fact the Placebo group saw a decrease in total sleep time from baseline to Day 56 suggesting that participants were awake for more time and participants couldn't sleep efficiently).

Onset Latency (Time required to fall asleep once the subject goes to bed): This analysis showed that participants who were administered KaraCalm™ saw a statistically significant decrease in onset latency at Day 14 and Day 56 compared to the placebo group. KaraCalm™ subjects had a mean onset latency time of 17.5 minutes at baseline and this decreased to 10.01 minutes at Day 14 and 5.25 minutes at Day 56.

Sleep Efficiency (Sound Sleep): Participants who were administered Karacalm™ experienced a statistically significant increase at Day 14 and Day 56 compared to the Placebo group.

WASO (Wake-up after sleep onset) minutes: KaraCalm™ group saw a statistically significant decrease at Day 14 and Day 56 compared to the Placebo group.

AWAKE (AWAKEINING): The KaraCalm™ group saw a statistically significant decrease compared to the placebo group at Day 14 and Day 56 in the frequency of awakenings suggesting that KaraCalm™ subjects experienced a more sound sleep as compared to Group B where only a slight reduction in number of awakenings was observed.

The KaraCalm™ group experienced a statistically significant decrease in the PSS, hsCRP and S Cortisol scores from baseline to the end of the study. The KaraCalm™ group outperformed the Placebo group in all four parameters.

### Safety Evaluation

The study comprised of two arms-Treatment Group KaraCalm™ and Placebo Group. The duration of intervention consumption was for 56 days and each participant consumed either one of the IPs or Placebo once daily after dinner.

There were no adverse events observed in this study.

There were no serious adverse events, or other significant adverse events.

Every participant in both groups had their vital signs-systolic blood pressure (SBP), diastolic blood pressure (DBP), pulse, respiration rate, and body temperature-measured at every visit. Descriptive statistics were applied to all available data for safety evaluations. The safety statistics were within normal ranges and did not show any significant differences within or between the groups.

At every study visit, the following parameters were recorded: temperature, pulse rate, respiration rate, and systolic and diastolic blood pressure. The safety limits for each visit were within normal range.

### Discussion

The study was designed to evaluate safety and efficacy of KaraCalm™-A Dietary supplement-novel herbal formulation to assess its properties towards managing stress and improving sleep among adults. After and the study approval by the ethics committee, the study was registered.

Healthy adults male and female subjects with age ranging from 18 to 54 years, free of psychiatric conditions and who were willing to provide written informed consent were enrolled in the study.

Each subject was provided with detailed procedure information and appropriate consent was obtained prior to the study procedures.

The primary and secondary objectives were clearly defined, and study outline of the trial was carried out with screening and randomization of the eligible subjects in the intervention and placebo group. Subjects were prescribed to consume 500 mg capsules once daily orally for 56 days.

The primary parameter assessed was Sleep analysis evaluated by using Acti Watch at along with perceived Stress Scale (PSS) scores.

These parameters were assessed using unpaired ‘t’ test and ANOVA with post hoc Tukey's test for both the study groups (KaraCalm™ and placebo).

Other efficacy parameters included Serum Cortisol and Hs CRP evaluated for both the groups.

Safety parameters such as systolic and diastolic blood pressure, temperature, pulse rate, respiratory rate was evaluated at all time visits and analyzed using one-way ANOVA with post hoc Tukey's test.

The primary outcome of Actigraphy study (Sleep Analysis) using Acti Watch and PSS Scores suggested that KaraCalm™ resulted in a beneficial and statistically significant improvement following its consumption.

Upon further analysis, the secondary outcomes relating to the Serum cortisol and Hs CRP showed that KaraCalm™ brought a statistically significant improvement compared to the placebo.

The changes seen in primary and secondary outcome were statistically significant indicating the positive efficacy of KaraCalm™.

The safety parameters assessment was done with clinical laboratory tests and vital signs.

The vital signs showed no variation from the normal thus making KaraCalm™, a safe medication.

With respect to PSS the KaraCalm™ group saw a statistically significant improvement compared to the placebo group at Day 14, Day 28 and Day 56.

For both Serum Cortisol and Hs CRP levels, the KaraCalm™ group saw a statistically significant improvement compared to the placebo group at Day 28 and Day 56.

There was no significant change in the safety analyses of the patients in intervention group when assessed from the start of the study to the end, thus indicating that KaraCalm™ was well tolerated and safe for consumption.

### CONCLUSIONS

The mean age in Group A was 31.37±9.59 years while in Group B it was 32.25±8.37 years.

For the KaraCalm™ group, there was increase in total sleep time and sleep efficiency and a decrease in sleep onset latency, WASO minutes, and frequency of awakenings indicating the effectiveness of KaraCalm™ in improving sleep quality.

There was decrease in PSS scores in KaraCalm™ group from baseline to end of the study indicating its effectiveness in reducing stress.

There was a significant reduction in Serum Cortisol and Hs CRP levels in KaraCalm™ group from baseline to end of the study, indicating the effectiveness of KaraCalm™ in reducing stress.

The KaraCalm™ group did not experience a change in their vital signs, thus indicating the safety of KaraCalm™.

Based on the overall results it appears that KaraCalm™ is a safe and effective dietary herbal supplement to manage stress and improve sleep quality.

TABLE 7
Comparison of Sleep Analysis Actigraphy between
Group A and Group B at different visits

| | | | Std. | Mean | |
|---|---|---|---|---|---|
| | Groups | Mean | Deviation | Difference | P Value |
| Bed time | Group A | 20:59:39 | 4:31:25 | −0:55:56 | .328 |
| Day 2 | Group B | 21:55:35 | 0:55:15 | | |
| Bed time | Group A | 21:34:39 | 1:07:18 | 0:30:38 | .601 |
| Day 14 | Group B | 21:04:01 | 4:37:00 | | |
| Bed time | Group A | 21:35:39 | 4:09:03 | −0:44:00 | .015* |
| Day 56 | Group B | 22:19:39 | 1:03:02 | | |
| Get up time | Group A | 5:22:08 | 0:48:38 | −0:12 | .406 |
| Day 2 | Group B | 5:34:19 | 0:51:48 | | |
| Get up time | Group A | 6:06:46 | 0:58:04 | 0:54:05 | .000* |
| Day 14 | Group B | 5:12:41 | 0:38:56 | | |
| Get up time | Group A | 6:54:57 | 1:24:36 | 2:12:21 | .000* |
| Day 56 | Group B | 4:42:35 | 1:02:04 | | |
| Time in bed | Group A | 7:17:28 | 0:53:37 | −0:21 | .189 |
| Day 2 | Group B | 7:38:43 | 0:56:41 | | |
| Time in bed | Group A | 8:26:25 | 1:51:20 | 0:59:40 | .019* |
| Day 14 | Group B | 7:26:45 | 0:44:12 | | |
| Time in bed | Group A | 9:25:18 | 1:34:53 | 1:57:44 | .00* |
| Day 56 | Group B | 7:27:33 | 1:07:06 | | |
| Total sleep | Group A | 5:46:59 | 1:00:14 | −0:20 | .291 |
| time Day 2 | Group B | 6:06:59 | 1:09:06 | | |
| Total sleep | Group A | 6:58:56 | 1:39:02 | 0:45:23 | .049* |
| time Day 14 | Group B | 6:13:33 | 0:47:16 | | |
| Total sleep | Group A | 8:01:42 | 2:02:25 | 2:17:52 | .000* |
| time Day 56 | Group B | 5:43:50 | 0:54:03 | | |
| Onset | Group A | 17.5071 | 6.70425 | −.22667 | .913 |
| latency Day | Group B | 17.7338 | 7.51488 | | |
| 2 | | | | | |
| Onset | Group A | 10.0104 | 4.16119 | −7.16208 | .000* |
| latency Day | Group B | 17.1725 | 7.26270 | | |
| 14 | | | | | |
| Onset | Group A | 5.2525 | 3.17142 | −12.38500 | .000* |
| latency Day | Group B | 17.6375 | 10.03973 | | |
| 56 | | | | | |
| Sleep | Group A | 77.3079 | 4.93797 | 1.79500 | .363 |
| efficiency | Group B | 75.5129 | 8.19545 | | |
| Day 2 | | | | | |
| Sleep | Group A | 81.8479 | 6.30837 | 5.49583 | .004* |
| efficiency | Group B | 76.3521 | 6.41565 | | |
| Day 14 | | | | | |
| Sleep | Group A | 86.5767 | 4.53612 | 9.02167 | .000* |
| efficiency | Group B | 77.5550 | 6.54002 | | |
| Day 56 | | | | | |
| WASO - | Group A | 104.9217 | 22.31936 | 47.34750 | .000* |
| Minutes Day | Group B | 57.5742 | 18.09829 | | |
| 2 | | | | | |
| WASO - | Group A | 75.6346 | 14.31237 | 17.94833 | .001* |
| Minutes Day | Group B | 57.6863 | 19.55622 | | |
| 14 | | | | | |
| WASO - | Group A | 60.7204 | 16.62589 | 9.42083 | .041* |
| Minutes Day | Group B | 51.2996 | 14.33775 | | |
| 56 | | | | | |
| #AWAKE | Group A | 26.00 | 4.95 | .64667 | .683 |
| Day 2 | Group B | 25.36 | 5.89 | | |
| #AWAKE | Group A | 18.55 | 5.68 | −6.33333 | .000* |
| Day 14 | Group B | 24.88 | 4.70 | | |
| #AWAKE | Group A | 12.32 | 5.32 | −12.11667 | .000* |
| Day 56 | Group B | 24.44 | 6.71 | | |

TABLE 8
Pairwise Comparison of Sleep Analysis
Actigraphy between Group A and Group B

| | | | Group A | | Group B | |
|---|---|---|---|---|---|---|
| | (I) | (J) | Mean | | Mean | |
| Dependent | Time | Time | Difference | P | Difference | P |
| Variable | Interval | Interval | (I-J) | Value | (I-J) | Value |
| Bed time | Day 2 | Day 14 | −0:34:59 | .743 | 0:51:34 | .536 |
| | | Day 56 | −0:35:59 | .731 | −0:24:03 | .872 |
| get up time | Day 2 | Day 14 | −0:44 | .050* | 0:21:37 | .323 |
| | | Day 56 | −1:32 | .000* | 0:51:43 | .003* |
| time in bed | Day 2 | Day 14 | −1:08:56 | .026* | 0:11:58 | .747 |
| | | Day 56 | −2:07:49 | .000* | 0:11:09 | .775 |
| total sleep | Day 2 | Day 14 | −1:11:57 | .033* | −0:06:33 | .918 |
| time | | Day 56 | −2:14:43 | .000* | 0:23:09 | .350 |
| onset | Day 2 | Day 14 | 7.49667 | .000* | .56125 | .971 |
| latency | | Day 56 | 12.25458 | .000* | .09625 | .999 |
| sleep | Day 2 | Day 14 | −4.54000 | .012* | −.83917 | .912 |
| efficiency | | Day 56 | −9.26875 | .000* | −2.04208 | .581 |
| WASO - | Day 2 | Day 14 | 29.28708 | 0.00* | −.11208 | 982 |
| Minutes | | Day 56 | 44.20125 | 0.00* | 6.27458 | 0.143 |
| #AWAKE | Day 2 | Day 14 | 7.46333 | .000* | .48333 | .956 |
| | | Day 56 | 13.68417 | .000* | .92083 | .848 |

Intragroup Comparison done using ANOVA with Post Hoc Tukey's (*=p < 0.05)
*The mean difference is significant at the 0.05 level.

TABLE 9
Comparison of PSS between Group A and
Group B at different time visits

| | | | Std. | Mean | P |
|---|---|---|---|---|---|
| | Groups | Mean | Deviation | Difference | Value |
| Day 2 PSS | Group A | 21.5833 | 1.31601 | .08333 | .830 |
| | Group B | 21.5000 | 1.35133 | | |
| Day 14 PSS | Group A | 18.5000 | 1.44463 | −3.16667 | .000* |
| | Group B | 21.6667 | 1.40393 | | |
| Day 28 PSS | Group A | 15.7083 | 1.87615 | −5.75000 | .000* |
| | Group B | 21.4583 | 1.55980 | | |
| Day 56 PSS | Group A | 12.8750 | 1.29590 | −7.37500 | .000* |
| | Group B | 20.2500 | 2.13154 | | |

TABLE 10
Pairwise comparison of PSS between Group A and Group B

| | | | Group A | | Group B | |
|---|---|---|---|---|---|---|
| | (I) | (J) | Mean | | Mean | |
| Dependent | Time | Time | Difference | P | Difference | P |
| Variable | Interval | Interval | (I-J) | Value | (I-J) | Value |
| PSS | Day 2 | Day 14 | 3.08333 | .000* | −0.1667 | 1.000 |
| | | Day 28 | 5.87500 | .000* | .04167 | 1.000 |
| | | Day 56 | 8.70833 | .000* | 1.25 | .059 |

TABLE 13
Comparison of Hs CRP between Group A and Group B

| | | | Std. | Mean | P |
|---|---|---|---|---|---|
| | Groups | Mean | Deviation | Difference | Value |
| Day 2 hs CRP | Group A | 2.5304 | .67480 | −.14917 | .428 |
| | Group B | 2.6796 | .61740 | | |
| Day 28 hs | Group A | 2.1954 | .68463 | −.44792 | .020* |
| CRP | Group B | 2.6433 | .60382 | | |
| Day 56 hs | Group A | .9142 | .10774 | −1.68208 | .000* |
| CRP | Group B | 2.5963 | .59033 | | |

TABLE 14
Pairwise Comparison of Hs CRP between Group A and B

| | | | Group A | | Group B | |
|---|---|---|---|---|---|---|
| | (I) | (J) | Mean | | Mean | |
| Dependent | Time | Time | Difference | P | Difference | P |
| Variable | Interval | Interval | (I-J) | Value | (I-J) | Value |
| hs CRP | Day 1 | Day 28 | .33500 | .102 | .03625 | .976 |
| | | Day 56 | 1.61625 | .000* | .08333 | .882 |

TABLE 15
Comparison of Serum Cortisol between Group A and Group B

| | | | Std. | Mean | P |
|---|---|---|---|---|---|
| | Groups | Mean | Deviation | Difference | Value |
| Day 2 S | Group A | 16.8546 | 2.21454 | .59792 | .312 |
| Cortisol | Group B | 16.2567 | 1.81638 | | |
| Day 28 S | Group A | 13.6054 | .91573 | −2.37250 | .000* |
| Cortisol | Group B | 15.9779 | 1.04710 | | |
| Day 56 S | Group A | 11.7246 | 1.18585 | −4.23125 | .000* |
| Cortisol | Group B | 15.9558 | 1.28249 | | |

TABLE 16
Pairwise Comparison of Serum Cortisol between Group A and B

| | | | Group A | | Group B | |
|---|---|---|---|---|---|---|
| | (I) | (J) | Mean | | Mean | |
| Dependent | Time | Time | Difference | P | Difference | |
| Variable | Interval | Interval | (I-J) | Value | (I-J) | P Value |
| S Cortisol | Day 2 | Day 28 | 3.24917 | .000* | .27875 | .776 |
| | | Day 56 | 5.13000 | .000* | .30083 | .744 |

While this disclosure includes specific examples, it will be apparent after an understanding of the disclosure of this application has been attained that various changes in form and details may be made in these examples without departing from the spirit and scope of the claims and their equivalents.

1. (canceled)

2. A composition comprising about 16% to about 24% by weight of Valeriana officinalis extract; about 12% to about 18% by weight of Passiflora incarnata extract; about 12% to about 18% by weight of Ocimum sanctum extract; about 12% to about 18% by weight of Ziziphus jujuba extract; about 12% to about 18% by weight of Rosmarinus officinalis extract; and about 16% to about 24% by weight of Nigella sativa extract.

3. The composition of claim 2 comprising about 20% by weight of Valeriana officinalis extract; about 15% by weight Passiflora incarnata extract; about 15% by weight of Ocimum sanctum extract; about 15% by weight of Ziziphus jujuba extract; about 15% by weight of Rosmarinus officinalis extract; and about 20% by weight of Nigella sativa extract.

4. The composition of claim 2 comprising as an active ingredient: (E)-3-[(1R,4S,7R,7aR)-1-hydroxy-3,7-dimethyl-2,4,5,6,7,7a-hexahydro-1H-inden-4-yl]-2-methylprop-2-enoic acid; (E)-3-[(1R,4S,7R,7aR)-1-acetyloxy-3,7-dimethyl-2,4,5,6,7,7a-hexahydro-1H-inden-4-yl]-2-methylprop-2-enoic acid; or (E)-3-[(4S,7R,7aR)-3,7-dimethyl-2,4,5,6,7,7a-hexahydro-1H-inden-4-yl]-2-methylprop-2-enoic acid.

5. The composition of claim 2 comprising as an active ingredient: (1S,2R,4aS,6aR,6aS,6bR,8aR,10S,12aR,14bS)-10-hydroxy-1,2,6a,6b,9,9,12a-heptamethyl-2,3,4,5,6,6a,7,8,8a,10,11,12,13,14b-tetradecahydro-1H-picene-4a-carboxylic acid.

6. The composition of claim 2 comprising as an active ingredient: a flavonoid comprising Vitexin.

7. The composition of claim 2 comprising as an active ingredient: (2R)-3-(3,4-dihydroxyphenyl)-2-[(E)-3-(3,4-dihydroxyphenyl) prop-2-enoyl]oxypropanoic acid.

8. The composition of claim 2, wherein the composition is included in a capsule, tablet, or pill.

9. A method of treating a sleep disorder in a human comprising: administering to a patient in need thereof a pharmaceutically effective amount of the composition of claim 2.

10. A method of treating a stress-related disorder in a human comprising: administering to a patient in need thereof a pharmaceutically effective amount of the composition of claim 2.

11. The composition of claim 2, wherein the composition is included in a liquid, cream, paste, gel, suspension, dispersion, solid, emulsion, aerosol, powder, tablet, capsule, or pill.
